(19) United States
(12) Patent Application Publication
(10) Pub. No.: US 2026/0144454 A1
(43) Pub. Date: May 28, 2026

(54) METHOD OF DETERMINATION OF ZERO FLOW LEVEL AND A STAND FOR PULMONARY FUNCTION TEST DEVICES

(71) Applicant: INOFAB SAGLIK TEKNOLOJILERI ANONIM SIRKETI, Çankaya, (TR)

(72) Inventors: Merthan OZTURK, Çankaya (TR); Kerem YASAR, Çankaya (TR); Ahmet OGUZ, Çankaya (TR); Burak SONMEZ, Çankaya (TR); Kadir Tayyip TASLICA, Çankaya (TR); Selim YONET, Çankaya (TR)

(73) Assignee: INOFAB SAGLIK TEKNOLOJILERI ANONIM SIRKETI, Çankaya, (TR)

(21) Appl. No.: 19/454,245

(22) Filed: Jan. 20, 2026

Related U.S. Application Data
(63) Continuation of application No. 17/623,626.

(30) Foreign Application Priority Data
Jan. 5, 2020 (TR) 2020/01738
Feb. 4, 2021 (TR) 2021/01698

(51) Int. Cl. A61B 5/087

(52) U.S. Cl. CPC A61B 5/087

(57) ABSTRACT

A method of determination of a zero flow level for pulmonary function test devices and a stand of determination of the zero flow level used for implementing the method are provided. The method of determination of the zero flow level enabling the zero flow level of the pulmonary function test devices to perform under suitable conditions and the stand of determination of the zero flow level used for implementing the method are developed.

### CROSS REFERENCE TO THE RELATED APPLICATIONS

This application is a continuation of U.S. application Ser. No. 17/623,626 filed on Dec. 29, 2021, which claims priority to International Application No. PCT/TR 2021/050100, filed on Feb. 4, 2021, which is based upon and claims priority to Turkish Patent Application No. 2020/01738 filed on Feb. 5, 2020, and claims priority to Turkish Patent Application No. 2021/01698 filed on Feb. 4, 2021, the entire contents of which are incorporated herein by reference.

### TECHNICAL FIELD

This invention is related to a method of determination of zero flow level for pulmonary function test devices and to a stand of determination of zero flow level that can be used for implementing this method.

### BACKGROUND

Various assemblies enabling the measurement of the airflow rate for evaluating the respiratory functions of a patient are disclosed in U.S. Pat. No. 7,618,235, U.S. Pat. No. 7,383,740, and US20110092840. The volume of breath inhaled and exhaled during inhalation or exhalation can also be determined along with measuring the airflow rate.

Also, some of the ultrasonic pulmonary function test devices are disclosed U.S. Pat. No. 5,419,326, U.S. Pat. No. 5,647,370, JP2013250254 or US2010145213 numbered documents. On the other hand, a spirometer developed by Inofab is disclosed in the Turkish Patent application numbered 2017/04582. The spirometer that is the subject matter of said application comprises a tube enabling the transceiver to operate without getting affected by the contaminants introducing the air channel and contained in the airflow.

One of the measurement techniques used in these devices is the first edge measurement technique. In this method, a signal consisting of an ultrasonic wave having one or more wavelengths is sent from a transmitter to a receiver. The timer, which is started at the moment when the transmitter sends the ultrasonic signal, is stopped at the point of the next zero interruption after the first half-wave created on the receiver, thereby a measurement is performed. This point of zero interruption occurs after the amplitude of the first half-wave created on the receiver is above a specific value. The ultrasonic pulmonary function test devices used widely perform the flow rate measurement by calculating the times of flight of the signal between two transceivers on a line intersecting the flow direction obliquely and by comparing these times of flight.

In order to enable the pulmonary function test devices to perform correct and accurate measurements, it is necessary to determine the zero flow level, i.e. to perform a measurement without any airflow, thereby storing the result of this measurement for correcting the measurements obtained with the future pulmonary function tests. For this purpose, it is necessary to start the zero flow level process after the user has warned about holding the device stable. Therefore, zero flow level cannot be determined safely by the personal users as well as causes workload and loss of time for the medical personnel.

EP3566647A1 numbered document describes the improvements related to the verification of calibration and accordingly, addresses the matter of interrogation of the measurement performed with the gases that are considered as inert. However, it is necessary to develop—the solutions for performing the measurement related to zero flow level without any airflow.

### SUMMARY

The aim of this invention is to develop a method of determination of zero flow level enabling the pulmonary function test devices to determine zero flow level easily and safely.

Another aim of this invention is to develop a method of determination of zero flow level allowing determining that the suitable conditions have been created for the determination of zero flow level, thereby the related measurement is performed automatically.

Yet another aim of this invention is to develop a stand of determination of zero flow level that can be used for implementing the developed method of determination of zero flow level.

By the developed—method and stand of determination of zero flow level, the zero flow level can be determined without causing a lot of work for the users, and the user-related errors are prevented. The invention provides advantages during the usage of pulmonary function test device s for personal use since the users have limited information and experiences regarding the medical devices and do not want to deal with the determination process of zero flow level, and furthermore facilitates the determination of zero flow level in pulmonary function test devices for clinical use, ensures that the measurement related to zero flow level is performed under the right conditions, enables the zero flow level determination to iterate frequently and alleviates the workload of medical personnel. By means of the female-male matching between the pulmonary function test device-seated to the stand and the stand, it is ensured that there is no airflow inside. Intrinsically the spirometers have two openings. Yet another aim of the invention is closing the opening closer to the bottom by seating to the stand as well as closing the other opening that is at the top of the device by a protrusion or a cavity in the stand such that it will close the opening of the device by the form of the stand itself

Similarly with a battery housing structure, it is possible to close the air inlet and outlet on the spirometer by pushing it on the spring to be seated in a housing with both lower and upper openings closed.

### BRIEF DESCRIPTION OF THE DRAWINGS

In the following the figures and the related descriptions for a better understanding of the method and stand of determination of zero flow level developed by this invention is given.

FIG. 1 is a schematic view of a pulmonary function test device whose zero flow level can be determined by the invention.

FIG. 2 is a flow chart of the method of determination of zero flow level according to the invention.

FIG. 3 is a schematic view of the stand of determination of zero flow level according to the invention.

FIG. 4 is a schematic view of the stand of determination of zero flow level according to the invention with a pulmonary function test device.

FIG. 5 is a perspective view of a pulmonary function test device-whose zero flow level can be determined by the invention.

FIG. 6 is a perspective view of the stand of determination of zero flow level according to the invention with a pulmonary function test device.

FIG. 7 is a perspective view of a pulmonary function test device whose zero flow level can be determined by the invention.

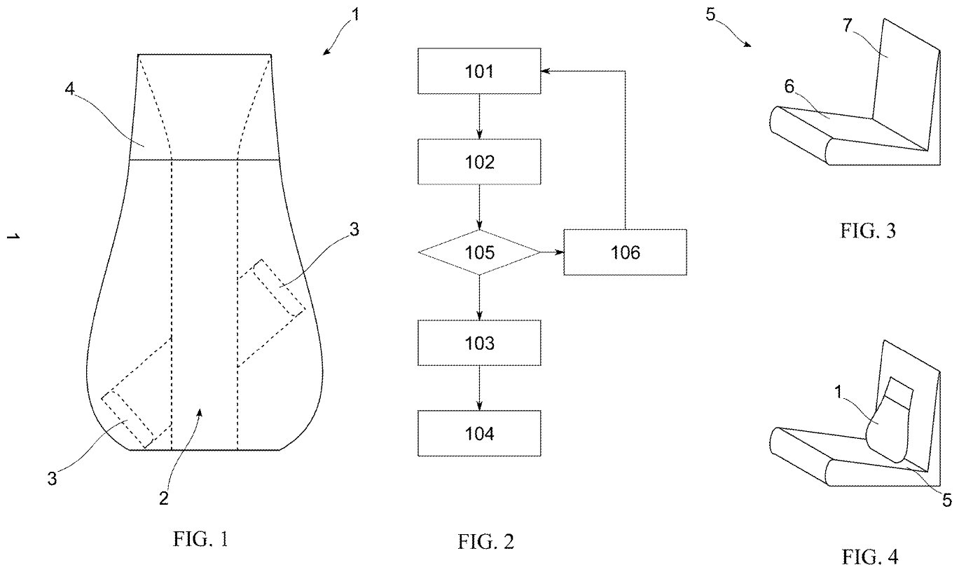

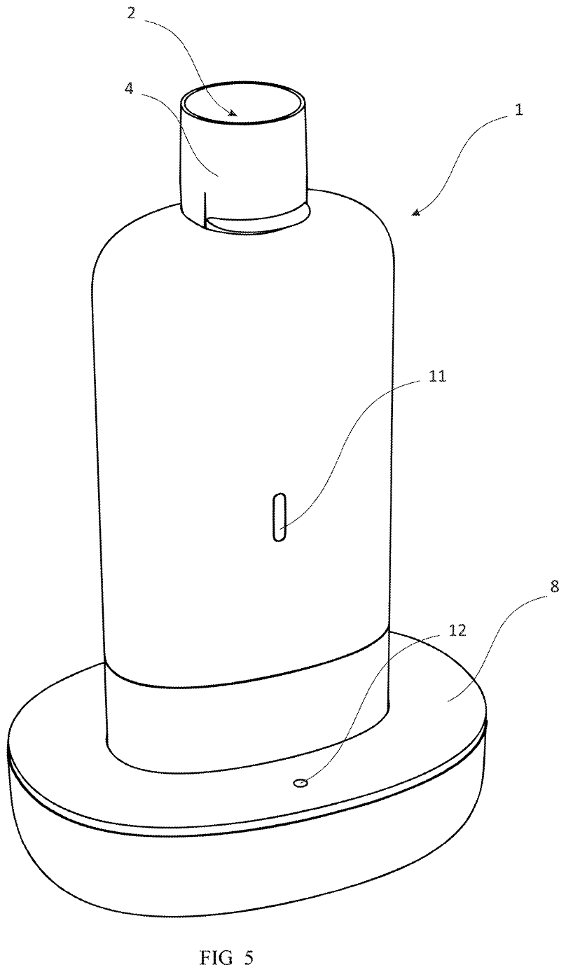

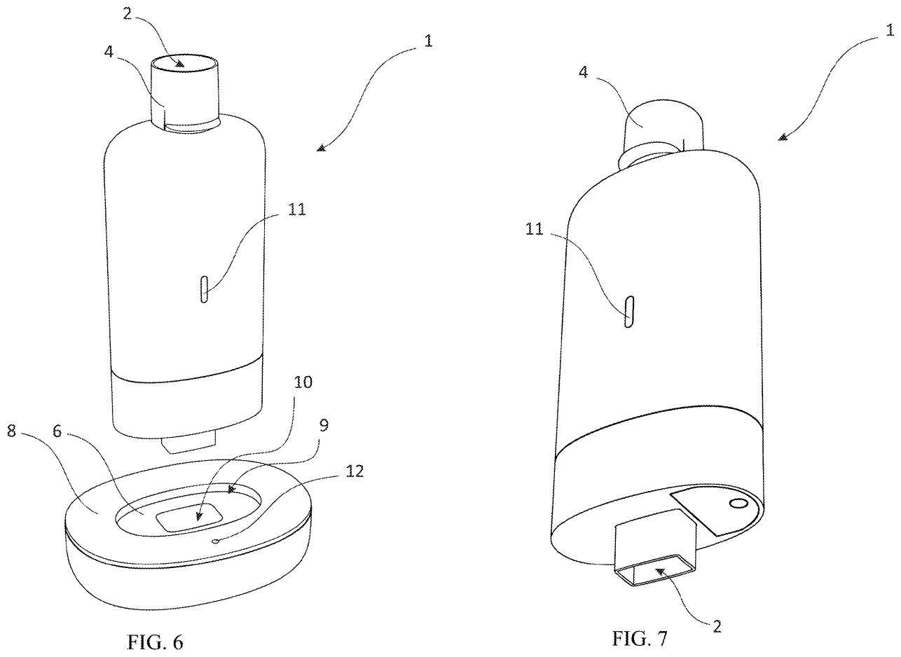

The pieces and parts in the figures are numbered for a better understanding of the method and stand of determination of zero flow level improved by this invention is given, the meaning of each number is given in the following.
1. pulmonary function test device
2. Airway
3. Sensor
4. Mouthpiece
5. Stand
6. Base
7. Support
8. Stand surface
9. Device seat
10. Mouthpiece seat
11. Device LED
12. Stand LED

### DETAILED DESCRIPTION OF THE EMBODIMENTS

A method of determination of a zero flow level that is the subject matter of the invention, providing the determination of the zero flow level of a pulmonary function test device (1) comprising sensors (3) positioned such that they can interact with an airway (2) to provide the measurement of time of flight of at least one signal by this airway (2) extending between two holes opening to the outer environment, essentially comprises the steps of (101) positioning the pulmonary function test device (1) such that one of the holes of the airway (2) is covered,

(102) at this stage, performing a measurement corresponding to a zero flow condition by operating the sensors (3),

(103) storing the measurement corresponding to a zero flow condition,

(104) using the measurement corresponding to the last stored zero flow condition in the measurements performed within the scope of pulmonary function tests.

The pulmonary function test devices (1) can be spirometers. The sensors (3) preferably are ultrasonic transceivers.

Two of the holes should interact with the outer environment in order to create an airflow inside the airway (2). The air flows that can occur when one of the holes is closed are at a negligible level under the expected use conditions.

In an embodiment of the invention, in step 103 a time of flight measurement—corresponding to zero flow condition is stored as a zero flow time of flight. In this case, in step 104 the time of flight is calculated after the zero flow time of flight is subtracted from the time of flight—read during the measurement.

In another embodiment of the invention, in step 103 a time of flight calculated by using the signal corresponding to zero flow condition is stored as a time of flight of zero flow. In this case, in step 104 the corrected time of flight is calculated by subtracting the time of flight of zero flow from the time of flight calculated as a result of the measurement.

The above-mentioned subtracting the zero flow signal or the time of flight of zero flow means the superposition of the negative of the value related to zero flow with the measurement result.

With the method of determination of zero flow level that is the subject matter of the invention, zero flow level of the pulmonary function test device (1) can be determined even in the case that a mouthpiece (4) coinciding with the airway (2) is attached. In an embodiment of the invention, in order to prevent the effects of the parts of the mouthpiece (4) intersecting with the line between the sensors (3) on the measurement to degrade the measurement. accuracy, it is necessary to implement the method of determination of zero flow level in a case that the mouthpiece (4) is attached to the pulmonary function test device (1).

In order to determine whether the mouthpiece (4) is attached correctly during the determination of zero flow level, in an embodiment of the invention, the steps of

(105) comparing the measurement corresponding to zero flow condition with the range of edge measurement results that can be obtained in the case that the mouthpiece (4) is attached correctly,

(106) producing an error notification if the measurement corresponding to zero flow condition is outside the range of edge measurement results that can be obtained in the case that the mouthpiece (4) is attached correctly,

are conducted between the steps 102 and 103. The range of edge—measurement results that can be obtained in the case that the mouthpiece (4) is attached correctly is a predefined range that is formed experimentally by taking into account the production tolerances of the mouthpiece (4). The different ranges of edge measurement results can be defined for the different mouthpiece (4) models.

Step 101 can be conducted by making one of the holes of the airway (2) of the pulmonary function test device (1) bear against a surface. In step 101 preferably, one of the holes of the airway (2) of the pulmonary function test device (1) is seated on the floor or a base (6) having a specific slope on the stand (5) of determination of zero flow level.

In an embodiment of the invention, when the determination of the zero flow level is required, a notification for positioning the pulmonary function test device (1) such that one of the holes of the airway (2) is covered can be delivered to the user. The need for determination of zero flow level can be deduced if a predefined period has passed after the determination of the last stored zero flow level, a predefined number of pulmonary function tests has performed after the determination of the last stored zero flow level or a deviation exceeding a predefined level is observed in the measurement results obtained with the pulmonary function tests.

Step 102 can be conducted by assuming that step 101 is conducted after waiting for a specific period following the notification is delivered to the user. Instead, step 102 can be conducted after detecting that step 101 is conducted.

In another embodiment of the invention, step 102 is conducted after detecting that step 101 1s conducted.

In another embodiment of the invention, step 102 is conducted after detecting that step 101 1s conducted, however in case that the conduction of step 101 cannot be detected or in case that the determination of zero flow level is required a notification is delivered to the user.

In step 101, whether the pulmonary function test device (1) is positioned such that one of the holes of the airway (2) is covered can be detected by receiving the related input from the user.

In step 101, in order to detect that the pulmonary function test device (1) has positioned such that one of the holes of the airway (2) is covered, in an embodiment of the invention, the orientation of the pulmonary function test device (1) is monitored and detecting that the pulmonary function test device (1) has positioned with an orientation such that one of the holes of the airway (2) is covered. For this purpose, the pulmonary function test device (1) can also cooperate with at least one orientation sensor. The orientation sensors can be situated on the pulmonary function test device (1) and/or in an external position. The orientation sensors have characteristics providing the determination of the orientation of the pulmonary function test device (1) by monitoring the movements of the pulmonary function test device (1), monitoring a reference signal or any other means, depending on the gravity affecting the pulmonary function test device (1). The orientation sensors can also distinguish between an orientation adopted by the pulmonary function test device (1) instantaneously during the movement and orientation by which the device is positioned in a stable manner. Thus, when the pulmonary function test device (1) is moving, the erroneous measurements related to zero flow level can be prevented. The orientation sensors have characteristics providing the determination whether the pulmonary function test device (1) is stable by monitoring the velocities, comparing a predefined range of time with the iterated measurements or any other means.

In an embodiment of the invention wherein the pulmonary function test device (1) comprises at least one orientation sensor, a measurement corresponding to zero flow can be triggered by detecting that the pulmonary function test device—(1) has positioned with an orientation corresponding to seating one of the holes of the airway (2) to the floor.

In another embodiment of the invention wherein the pulmonary function test device (1) comprises at least one orientation sensor, a measurement corresponding to zero flow can be triggered by detecting that the pulmonary function test device (1) has positioned with an orientation corresponding to seating one of the holes of the airway (2) to the base (6) of a stand (5) of determination of zero flow level.

A stand (5) of determination of zero flow level according to the invention comprises essentially a base (6) having a specific slope against which the pulmonary function test device (1) can be seated such that one of the holes of the airway (2) is covered. The stand (5) of determination of zero flow level can also comprise at least one support (7) connected to the base (6) in order to seat the pulmonary function test device (1) safely to the base (6). The orientation sensors can be situated on the stand (5) of determination of zero flow level or respectively, on the pulmonary function test device (1) and stand (5) of the zero flow level.

The support (7) can be formed of a surface against which the pulmonary function test device (1) is beared such that it is seated in the base, of a protrusion situated on the base (6) and seated in the inner surface of the airway (2) or one or more other structure holding the pulmonary function test device (1) appropriately on the base.

In an embodiment of the invention, a seat in which the pulmonary function test device (1) can be seated only with specific orientations on the support (7) and/or the base (6) can be provided. In this case, the orientation sensor can be formed only of the elements detecting the presence of the pulmonary function test device (1) inside the seat.

The orientation sensors can be formed of electrical contacts or switch which are situated mutually respectively on the stand (5) of zero flow level and on the pulmonary function test device (1) and which are associated with a recess and a protrusion engaging with each other when the pulmonary function test device (1) is in a specific orientation.

A stand (5) of determination of zero flow level according to the invention used with the chargeable respiratory test devices (1) can also act as a charging station. In this case, the charging elements can also act as an orientation sensor, i.e. determining that the pulmonary function test device (1) has seated to the stand (1) of determination of zero flow level with an orientation that will trigger the measurement for determining zero flow level can be detected by establishing a connection between the electrical contact performing the charging function or by establishing an inductive connection between the coilings performing the charging function.

A stand (5) of determination of zero flow level according to the invention can also act as a base station establishing communication between the pulmonary function test device (1) and a terminal or a server or as a back-up point enabling the pulmonary function test results to be stored. By means of providing wireless communication between the stand (5) of determination of zero flow level and pulmonary function test device (1), the communication between the stand (5) of determination of zero flow level and pulmonary function test device (1) can be maintained continuously.

Alternatively to the embodiment of the invention disclosed above, the pulmonary function test device (1) can be seated in the device seat (9) that is an opening formed on the stand surface (8) without being placed on this stand surface (8) forming the top of the stand (5). By means of the device seat (9), it is possible to carry the pulmonary function test device (1) in a more stable way and prevent the air ingress-egress in the mouthpiece (4).

In order to enable the pulmonary function test device (1) to prevent the mouthpiece (4) to be subjected to the undesired turbulences arising from the device body while the patient inhales and exhales, the mouthpiece (4) stands more outside relative to the base level of the body. The excess part of this mouthpiece extending outside does not allow the pulmonary function test device (1) to stand alone. however, the user should position the device on the stand (5) vertically.

The mouthpiece seat (10) situated in the middle of the device seat (9) on the stand (5) forms another space into which the mouthpiece (4) extending outside is inserted. It will be possible to interrupt in a staggered way the air flows inside the inner volume of the stand (5) created with the device seat (9) as well as the air flows created inside the mouthpiece (4) in the mouthpiece seat (10).

The test operation for determining zero flow level can be performed by seating the pulmonary function test device (1) in the device seat (9) on the stand (5). Whether the device is on the stand (5) can be determined as mentioned above with a slope or with elements such as a switch circuit as well as it can be deduced by using a magnet or hall effect sensors or proximity sensor without a contact. By placing any of these sensors to the base of the pulmonary function test device (1) or to a position near the top of the stand (5) with or without an orientation, it can be detected that the device is on the stand (5).

In the healthcare organizations where a plurality of pulmonary function test device s (1) and the related stand (5) are co-existent, it is likely to confuse the stands (5) with the devices attached thereto. In case that this possibility is experienced, the main function of the stand, i.e. the function of measuring the ambient temperature and humidity as well as the function of providing the ambient variables to be taken into account during the measurements to be performed by the pulmonary function test device (1) can be degraded. Since the ambient temperature and humidity will cause variances in the volume and intensity of the air inhaled or exhaled by the patient, the determination of zero flow level and subsequent measurements should be calibrated relative to the temperature and humidity values.

In case that the ambient temperature or humidity determined by a different stand (5) is used in the calibration of another pulmonary function test device (1) that is not matched therewith, it is possible to calculate the measurement results erroneously due to the ambient differences. Therefore, visual warnings such as co-flashing, sequentially flashing, intermittent flashing, and synchronous flashing of a device LED (11) and a stand LED (12) will be given, after the pulmonary function test device (1) has placed on the stand (5) and/or after the zero flow test has started and/or during the matching process and/or as long as the pulmonary function test device (1) is situated on the stand (5).

Since whether the pulmonary function test device (l)is placed on the stand (5) by the operator, doctor, medical personnel is matching correctly, is important in terms of the measurement of the necessary environment variables for the correct measurement that is the main object of the invention, eliminating the matching errors will also eliminate the occurrence of erroneous measurement.

Therefore, the condition of the device LED (11) or stand LED (12) is flashing in a specific order will indicate the followings to the user;
the pulmonary function test device (1) is seated in a location on the stand (5),
the air ingress into the mouthpiece (4) is blocked,
the stand (5) and the pulmonary function test device (1) are matched.

What is claimed is:

1. A stand for determination of zero flow level of a pulmonary function test device, the stand comprising:
a base contoured to position the pulmonary function test device in a predetermined orientation such that at least one opening of an airway of the pulmonary function test device is covered by the base and airflow through the airway of the pulmonary function test device is prevented.

2. The stand of claim 1, wherein the contour of the base includes:
a sloped surface on which the pulmonary function test device is seated; and
at least one support surface extending from the sloped surface, wherein the sloped surface and the at least one support surface are configured to position the pulmonary function test device in the predetermined orientation such that, when the pulmonary function test device is seated on the sloped surface in the predetermined orientation, the at least one opening of the airway bears against the sloped surface such that the at least one opening of the airway is covered by the sloped surface.

3. The stand of claim 2, wherein the sloped surface comprises at least one protrusion configured to be positioned in the at least one opening of the airway such that, when the pulmonary function test device is seated on the sloped surface, the pulmonary function test device is positioned in the predetermined orientation and the at least one opening of the airway is covered.

4. The stand of claim 2, wherein the stand further comprises at least one sensor configured to determine whether the pulmonary function test device is positioned on the stand in the predetermined orientation.

5. The stand of claim 4, wherein the at least one sensor comprises at least one of:
one or more hall effect sensors;
one or more proximity sensors; and
one or more switches.

6. The stand of claim 1, wherein the pulmonary function test device comprises a rechargeable pulmonary function test device, and wherein the stand further comprises at least one charging element configured to charge the rechargeable pulmonary function test device via at least one of a wired or wireless electrical connection between the rechargeable pulmonary function test device and the stand.

7. The stand of claim 6, wherein the stand is configured to determine a positioning of the pulmonary function test device in the predetermined orientation based on whether the pulmonary function test device is in wired or wireless electrical connection with the stand.

8. The stand of claim 1, further comprising at least one light-emitting-diode (LED) indicator disposed on the stand and configured to provide one or more visual warnings to a user.

9. The stand of claim 1, wherein the contour of the base includes:
a stand upper surface; and
a recessed seat surface disposed within the stand upper surface and recessed from the stand upper surface, such that the stand upper surface and the recessed seat surface form a device seat configured to receive at least a portion of a body of the pulmonary function test device and to position the pulmonary function test device in the predetermined orientation such that, when the pulmonary function test device is seated in the device seat, the at least one opening of the airway bears against the recessed seat surface such that the at least one opening of the airway is covered by the recessed seat surface.

10. The stand of claim 1, wherein the contour of the base includes:
a stand upper surface;
a recessed seat surface disposed within the stand upper surface and recessed from the stand upper surface, such that the stand upper surface and the recessed seat surface form a device seat configured to receive at least a portion of a body of the pulmonary function test device; and
a device protrusion seat disposed within the recessed seat surface and extending from the recessed seat surface into the base, the device protrusion seat configured to receive at least one protrusion extending from the body of the pulmonary function test device, wherein the at least one protrusion extending from the body of the pulmonary function test device defines the at least one opening of the airway, and wherein the device protrusion seat is further configured to position the pulmonary function test device in the predetermined orientation such that, when the at least one protrusion extending from the body of the pulmonary function test device is positioned within the device protrusion seat, the at least one opening of the airway is covered.

11. The stand of claim 9, wherein the base further comprises at least one sensor configured to determine whether the pulmonary function test device is seated in the device seat such that the pulmonary function test device is positioned in the predetermined orientation.

12. The stand of claim 10, wherein the base further comprises at least one sensor configured to determine whether the at least one protrusion extending from the body of the pulmonary function test device is seated in the device protrusion seat such that the pulmonary function test device is positioned in the predetermined orientation.

13. A pulmonary function test device comprising:
a body defining an airway extending through the body from a first end of the body to a second end of the body, the airway defining a first opening at the first end of the body and a second opening at the second of the body;
a stand configured for determination of zero flow level, the stand comprising:
a base contoured to position the body in a predetermined orientation such that at least one of the first opening or the second opening is covered by the base and airflow through the airway of the body is prevented.

14. The pulmonary function test device of claim 13, wherein the contour of the base includes:
a sloped surface on which the pulmonary function test device is seated; and
at least one support surface extending from the sloped surface, wherein the sloped surface and the at least one support surface are configured to position the body in the predetermined orientation such that, when the body is seated on the sloped surface in the predetermined orientation, at least one of the first opening or the second opening bears against the sloped surface such that the at least one of the first opening or the second opening is covered by the sloped surface.

15. The pulmonary function test device of claim 14, wherein the sloped surface comprises at least one protrusion configured to be inserted in at least one of the first opening or the second opening such that, when the body is seated on the at least one protrusion, the body is seated on the sloped surface in the predetermined orientation and the at least one of the first opening or the second opening is covered.

16. The pulmonary function test device of claim 13, the pulmonary function test device further comprising at least one sensor configured to determine whether the body is positioned on the stand in the predetermined orientation.

17. The pulmonary function test device of claim 13, wherein the contour of the base includes:
a stand upper surface; and
a recessed seat surface disposed within the stand upper surface and recessed from the stand upper surface, such that the stand upper surface and the recessed seat surface form a device seat configured to receive at least a portion of the body and to position the body in the predetermined orientation such that, when the body is seated in the device seat, the at least one of the first opening or the second opening bears against the recessed seat surface such that the at least one of the first opening or the second opening is covered.

18. The pulmonary function test device of claim 13, wherein:
the body further comprises a first airway extension extending from the first end of the body and defining the first opening of the airway and a second airway extension extending from the second end of the body and defining the second opening of the airway; and
the contour of the base includes:
a stand upper surface;
a recessed seat surface disposed within the stand upper surface and recessed from the stand upper surface, such that the stand upper surface and the recessed seat surface form a device seat configured to receive at least a portion of the body; and
a device protrusion seat disposed within the recessed seat surface and extending from the recessed seat surface into the base, the device protrusion seat configured to receive at least one of the first airway extension or the second airway extension and to position the body in the predetermined orientation such that, when the at least one of the first airway extension or the second airway extension is positioned within the device protrusion seat, the at least one of the first opening or the second opening is covered.

19. The pulmonary function test device of claim 17, wherein the base further comprises at least one sensor configured to determine whether the body is seated in the device seat such that the body is positioned in the predetermined orientation.

20. The pulmonary function test device of claim 18, wherein the base further comprises at least one sensor configured to determine whether the at least one of the first airway extension or the second airway extension is seated in the device protrusion seat such that the body is positioned in the predetermined orientation.
